(19) United States
(12) Patent Application Publication
(10) Pub. No.: US 2002/0198443 A1
(43) Pub. Date: Dec. 26, 2002

(54) Method and device for measuring blood sugar level

(76) Inventor: Choon Meng Ting, Singapore (SG)

Correspondence Address: Ladas & Parry, 26 West 61 Street, New York, NY, 10023, US

(21) Appl. No.: 10/150,766

(22) Filed: May 17, 2002

(30) Foreign Application Priority Data
Jun. 26, 2001 (SG) 200103981-7

(51) Int. Cl. A61B 5/00

(52) U.S. Cl. 600/323

(57) ABSTRACT

The invention provides a device for measuring blood sugar level in vivo, comprising means to generate a waveform signal derived from the systolic and diastolic cycle in an artery or capillary. It includes means to trigger a measurement of blood sugar level in the artery or capillary by non-invasive means in accordance with the waveform signal. The means to generate a waveform signal corresponding to the systolic and diastolic cycle may comprise an oximeter and the non-invasive measurement of blood sugar level may be performed by measuring the absorption of selected wavelengths of light transmitted by a light source.

### FIELD OF THE INVENTION

The invention relates to a method and device for measuring a user's blood sugar level. In particular, the method and device is non-invasive and is capable of measuring the user's blood sugar level continuously.

### BACKGROUND AND PRIOR ART

Traditionally, a person's blood sugar level is measured by a fine pin prick on the finger or blood drawn from the person's veins. However, one disadvantage of this method is that it is invasive.

Furthermore, the measurement of blood sugar level traditionally involves both capillary and venous blood. The present inventors have recognised that the source of blood sugar that would adversely affect a person's organs and cause organ damage and tissue perfusion is the blood at the capillary end of arterial blood vessels. That is, the region before glucose in the blood is released to the tissue.

Therefore, the measuring of a person's blood sugar level at the venous end may not be reflective of the true picture of the effects of target organ damage. For example, during an episode of hypoglycemia, the effects of the episode could actually occur at a higher level than the blood sugar level measured from the venous blood. That is also a possible reason why long-term complications of Neuropathy, Angiopathy and Nephopathy have not been eradicated but only postponed.

In order to measure the arterial blood sugar level at the capillary end of blood vessels, one should be able to capture the timing of the arterial pulse. The blood arriving at the arterial blood vessels is of a pulsative nature, according to the systolic and diastolic cycles of the heartbeat, unlike that of venous blood. The true level of the blood sugar level of the arterial blood would be at the height of the pulsation.

There are various patents submitted for measuring the blood sugar level non-invasively. Some of the disadvantages associated with these patents include:

(1) The equipment is not sufficiently portable to be used for monitoring blood sugar level at one's home and in particular, to allow continuous monitoring.

(2) The costs are too high due to the techniques used.

(3) They are too complex to operate, and require technicians and a laboratory to support the equipment.

(4) The methods use optical wavelengths beamed through the skin and soft tissue but a problem with data accuracy arises due to soft tissue interference. Therefore the differences in the penetration of tissue and absorption differences from various skin types reduce the accuracy with which the optical wavelengths can be measured.

### SUMMARY OF THE INVENTION

The invention seeks to alleviate at least some of the disadvantages associated with the prior art. It is an object of the invention to provide a method of portable, continuous and non-invasive measurement of blood sugar.

Preferably, the measurement of blood sugar level is made at the arteriole end of capillaries (pre-capillary), and timed to correspond to the systolic pulsation at the fingernail bed, using a pulse oximeter waveform as a gate control and trigger. By determining the capillary blood sugar level before blood sugar is utilized by the tissue, the net amount of sugar used after passing through the tissue can be measured. These blood sugar levels are “direct” effectors of end organ damage and related to insulin resistance, such that the data is potentially useful for determining medical indications.

According to the first aspect of the invention, the invention provides a device for measuring blood sugar level in vivo, comprising means to generate a waveform signal derived from the systolic and diastolic cycle in an artery or capillary, and means to trigger a measurement of blood sugar level in the artery or capillary by non-invasive means in accordance with the waveform signal.

Preferably, the means to generate a waveform signal corresponding to the systolic and diastolic cycle comprises an oximeter. It is preferable that the trigger means is set to trigger a measurement of blood sugar level when the waveform signal is at its highest and lowest as determined by the oximeter.

The non-invasive measurement of blood sugar level may be performed by measuring the absorption of selected wavelengths of light transmitted by a light source. It is also preferable that the light source is adapted to transmit light at two wavelengths capable of being absorbed by blood sugar. The light source may be adapted to transmit light at two wavelengths at or between 1500 nm and 2400 nm.

Preferably, the or each light source comprises a diode. Further, the device may include a light source adapted to transmit light at a control wavelength.

Preferably, the device includes a display device to display the blood sugar level. The display device may comprise a watch. Preferably, the device is particularly adapted for use on a finger or toe of a user.

Further, the oximeter is preferably a transmissive oximeter or a reflective oximeter.

According to a second aspect of the invention, the invention provides a method of measuring blood sugar level in vivo, comprising generating a waveform signal derived from the systolic and diastolic cycle in an artery or capillary of a subject and triggering measurement of blood sugar level in the artery or capillary in accordance with the waveform signal by non-invasive means.

The step of generating a waveform signal is preferably performed with an oximeter. The non-invasive means may comprise measuring the absorption of selected wavelengths of light.

The method may preferably also include the steps of triggering measurement of blood sugar level against a control when the waveform signal is at its highest, then triggering measurement of blood sugar level against a control when the waveform signal is at its lowest and calculating the difference between the values obtained.

Preferably, the method of measuring blood sugar level in vivo comprises the use of a device described as aforesaid.

It will be convenient to hereinafter describe the invention in greater detail by reference to the accompanying drawings which illustrate one embodiment of the invention. The particularity of the drawings and the related description is not to be understood as superseding the generality of the broad identification of the invention as defined by the claims.

### BRIEF DESCRIPTION OF THE DRAWINGS

FIG. 1 is a schematic illustration of the passage of blood from the arteries to the capillaries, feeding the target organs, and the exit thereof from the capillaries to the veins.

FIG. 2(a) is a cross-sectional illustration of the tip of a finger showing the subungal ridges and capillary columns extending adjacent a fingernail.

FIG. 2(b) is a plan representation of the arrangement of capillaries in a fingernail bed.

FIG. 3 illustrates an oximeter according to the preferred embodiment of the invention placed on a hand, together with a display device in the form of a watch worn on the wrist of the user.

FIG. 4 is a cross-sectional view of a first detailed embodiment of the present invention showing a finger inserted into an oximeter wherein a light source and a receptor are on opposite sides of a finger.

FIG. 5 is a conceptual illustration of a second detailed embodiment of the invention wherein a light source and a receptor are on the same side of a finger.

FIG. 6 is a cross-sectional view of a finger tip illustrating the angles at which the light source is beamed into the finger nail bed and reflected into a receiver according to the second embodiment of the invention.

FIG. 7 is an example of a waveform obtained using the oximeter according to the preferred embodiment of the invention.

FIG. 8 is a sample calibrator usable with the invention.

FIG. 9 is a flowchart showing the procedure of using an oximeter to determine the peak logic gate when the blood-sugar levels in the artery or capillary are at their highest.

FIG. 10 is a flowchart showing the procedure for obtaining readings from the absorption of light beams.

### DETAILED DESCRIPTION OF THE PREFERRED EMBODIMENT OF THE INVENTION

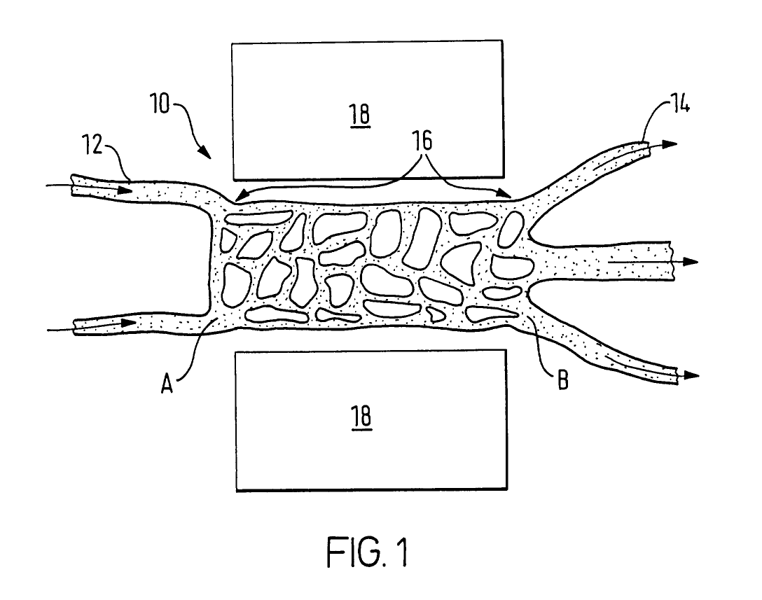

FIG. 1 is a schematic illustration of the passage of blood from the arteries to the capillaries, feeding the target organs, and the exit thereof from the capillaries to the veins. It demonstrates schematically the absorption of blood glucose by target organs, giving rise to the difference in blood sugar level between arterial and venous blood. Arterial blood arrives from the arterial blood vessels 12 and enter the blood sugar absorption region 10, which includes the capillaries 16 located near to the target organs 18, such as the kidneys, brain and heart. Blood sugar is absorbed into the target organs 18 and the blood exits the capillaries 16 to the venous blood vessels 14.

Referring to FIG. 1, blood enters the capillaries 16 at point A and exits at point B. The difference between the blood sugar levels at point A and point B would be equivalent to the amount of blood sugar consumed or extracted by the tissue of the body.

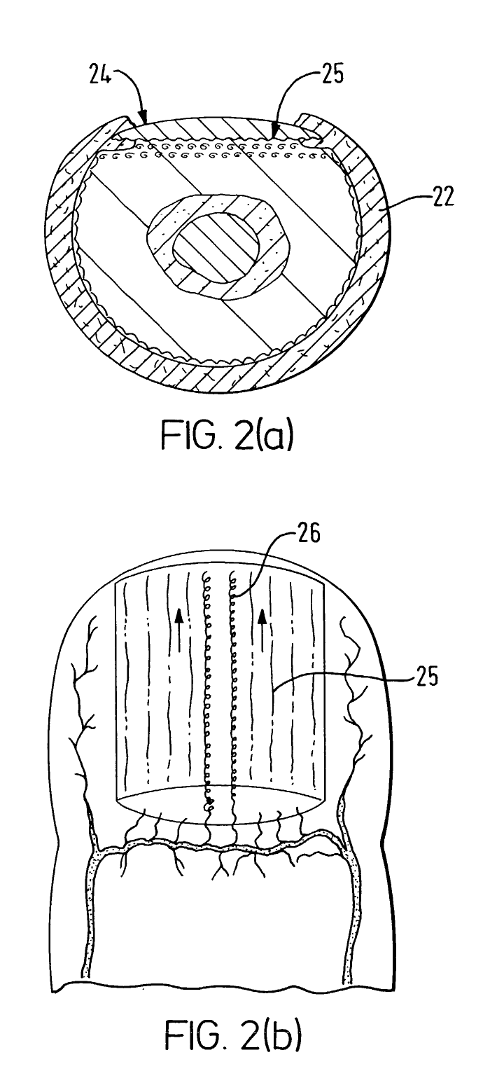

FIG. 2(a) is a cross-sectional illustration of the tip of a finger showing the subungal ridges and capillary columns extending adjacent a fingernail. FIG. 2(b) is a plan representation of the arrangement of capillaries in a fingernail bed. The fingernail bed is used for measuring the blood sugar levels at the arteriole end of the capillaries according to the preferred embodiment of the invention because of its unique anatomical arrangement.

Close examination of the structural arrangement of the area of the fingernail 24 will reveal that there are longitudinal ridges 25 that run in columns from the lunula distal to the hyponychierm. The undersurface of the nail (subungal epidermal ridges) contain ridges 25 that correspond to the longitudinal columns observed externally. This fits in a “tongue-in-groove” fashion with the nail bed 24. In between these grooves run the capillaries 26 which are spirally wound and radiate from the arcuate of arterioles that lie at the base of the nail. This is particularly visible at the distal one-third of the nail, which produces pink lines normally seen through the nail about 4 mm proximal to the tip of the finger.

Furthermore, penetration of light through the fingernail is relatively constant, unlike the penetration of light through the skin, which may vary according to factors such as the movement of the user. It also provides for a firm and solid surface for the light source to be emitted in a stable manner and detected. Different fingernails can be used at different times, which avoids the problem of skin irritation which would occur if the same site were used all the time. The properties of the nail surface thus make it an excellent site for optical work. It will be appreciated that a toenail has similar properties and can also be used for the measurement.

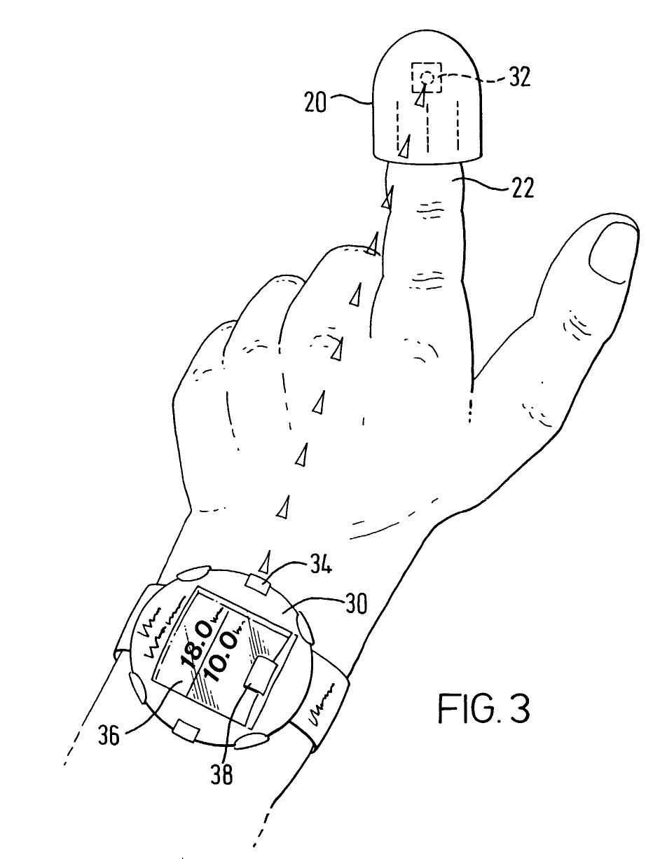

FIG. 3 illustrates an oximeter according to the preferred embodiment of the invention placed on a hand, together with a display device in the form of a watch worn on the wrist of the user. The oximeter 20 (such as a pulse-oximeter) is used as a gate control to trigger the emissions of selected wavelengths of light at the height of the arterial pulsation, ie. at the systolic cycle, or when the capillaries are filled at the nail bed. The oximeter 20 is shaped as a cap or finger-glove and is inserted onto the finger 22 of a user. The oximeter 20 has a transmitter 32 to transmit the readings obtained from the oximeter to a display device 30.

In FIG. 3, the display device 30 is in the embodiment of a wrist-watch, although other embodiments are possible. The display device 30 has a receptor 34 to receive the signals containing readings transmitted by the transmitter 32. The signals may be sent by a communications cable, but with suitable modification, wireless signals utilising technology such as infra-red or blue-tooth technology may be applied instead. In the illustrated embodiment in FIG. 3, the display device 30 has a display 36 to show the blood sugar readings. The display device may also include a microprocessor as well as print circuit board, high pass filter and amplifier, to process the readings obtained. Since the display device 30 may optionally function as a watch, a button 38 could be included on the display device 30 to indicate the blood sugar readings on the display 36 when the button 38 is pressed.

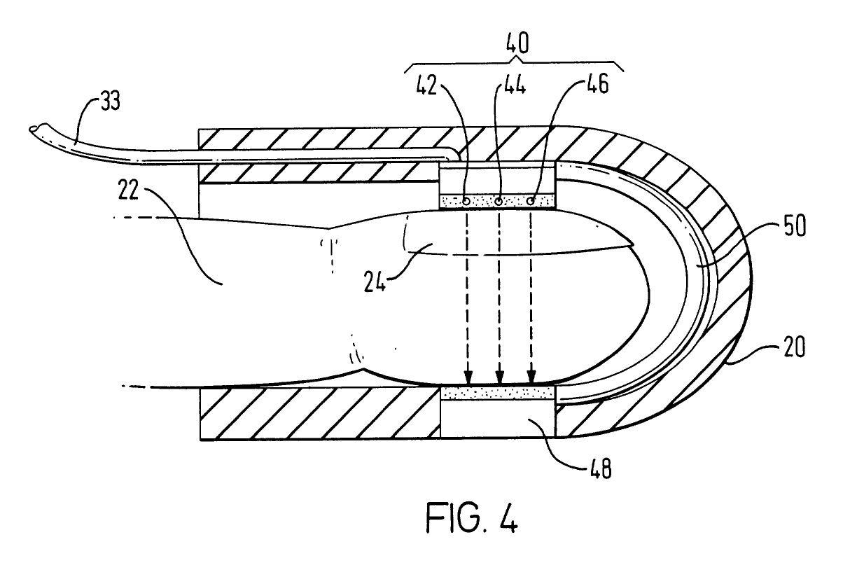

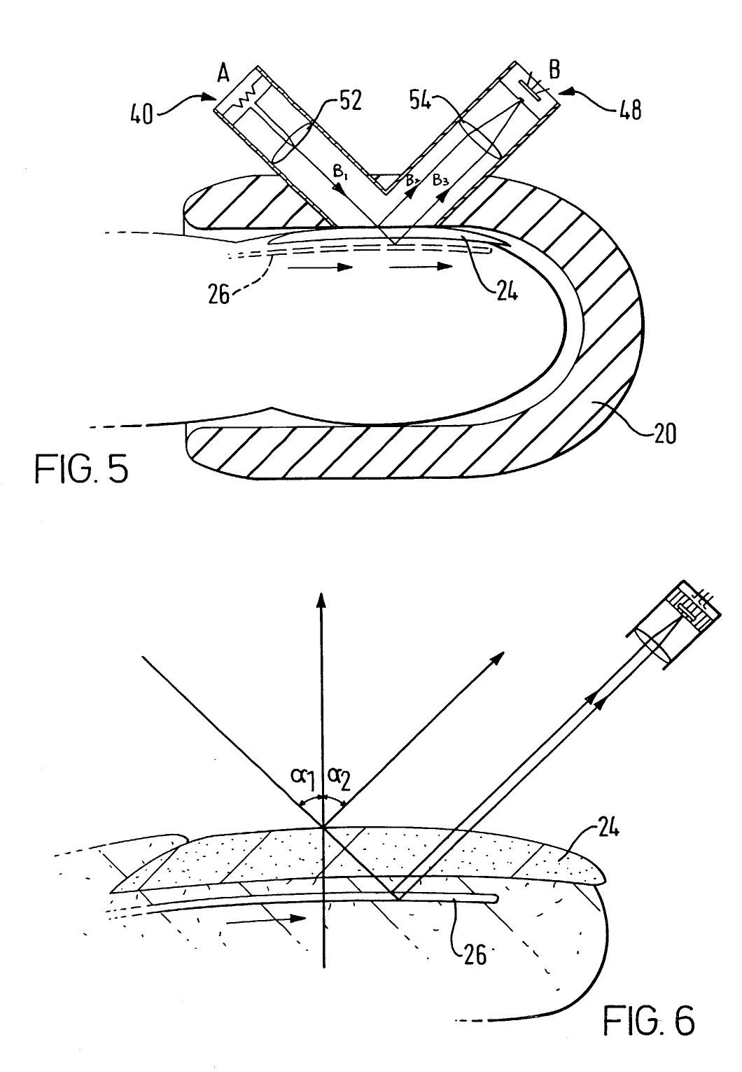

The method and principle of blood sugar measurement according to the preferred embodiment of the invention will now be described in more detail. FIG. 4 is a cross-sectional view of a first detailed embodiment of the present invention showing a finger inserted into an oximeter wherein a light source and a receptor are on opposite sides of a finger.

The oximeter 20 measures the PaO₂ (partial pressure of oxygen) percentage level in the blood at the fingernail bed. It also produces a waveform signal according to the systolic and diastolic cycle of the arterial pulse to ascertain when the blood sugar levels are at their maximum or minimum. In relation to the measurement of blood sugar using light, glucose molecules in the blood are able to absorb certain ranges of wavelengths of light. In vivo, there is a wide range of absorption, and it is partly due to interference by the tissue or bone. However, to improve accuracy and selectiveness of blood glucose, two or more wavelengths of light are selected at the input source. A third source of light for which the wavelength is not absorbed at all by glucose is chosen as a control.

The oximeter 20 illustrated is in the form of a finger glove, preferably made of rubber, mounted onto a fingernail. There is a light source 40 which emits three different wavelengths of light. One wavelength corresponds to a wavelength 42 capable of being absorbed by oxy-haemoglobin, and the other two wavelengths 44,46 are capable of being absorbed by glucose or blood sugar. The oximeter 20 is connected to a display device 30 by a cable 33 or other means as mentioned above for data transfer. For provision of power supply to the oximeter and light source, a cable is preferred.

The three wavelengths of light 42, 44, 46 are emitted from the light source 40 to penetrate the user's fingernail 24. The light beams 42, 44, 46 pass through the fingernail 24, tissue of the finger 22 and emerge on the opposite side of the finger 22. The light beams with different wavelengths 42, 44, 46 are detected by a light receptor 48 to measure the amount of each beam of light to penetrate the finger 22. A linking cable 50 may be included linking the light source 40 to the light receptor 48.

The procedure that a user may follow to measure his blood sugar level is now described.

A calibrator, which may be a standard coloured pad in the shape of the tip of a finger, is used for the purpose of calibrating the apparatus and verifying that it is in working condition.

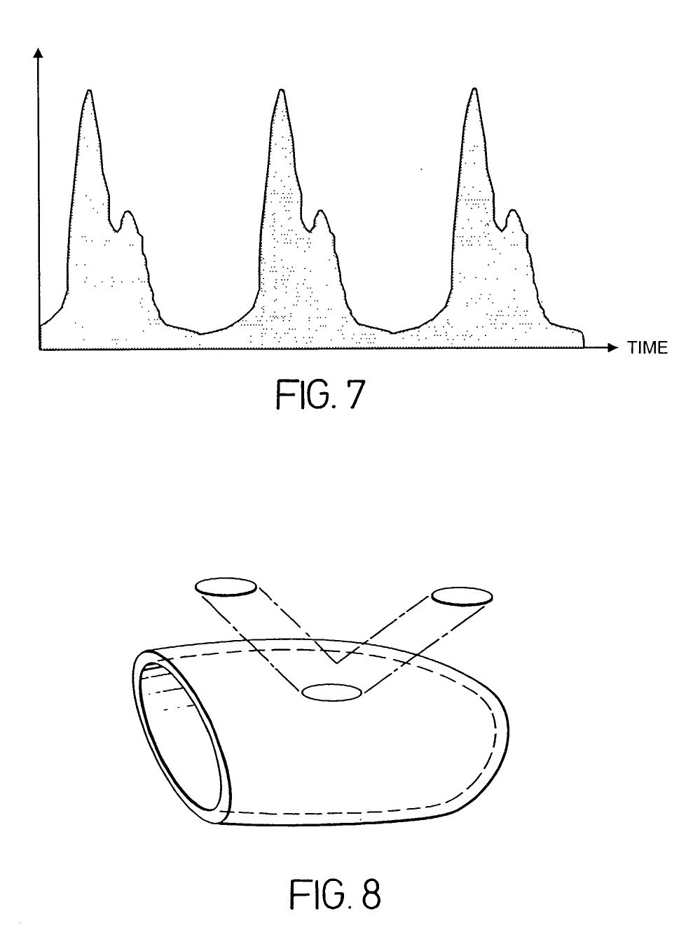

After calibration, the device is gloved onto the tip of the finger, which should be a finger with fingernail that is sufficiently clear for light to pass through. An oximeter source 20 would be the first part of the device to be triggered. The oximeter procedures a waveform signal consisting of peaks and troughs (see FIG. 7).

In the design of the logic gate, the arterial pulse waveform is first collected for a period of 10 to 15 seconds. This data is captured into the microprocessor by using a sampling time of say, 32 readings a cycle whereby the flow of the capillaries causes a change in the electrical signals as the systolic and diastolic cycle alternates. This sampling time is more than sufficient for plotting an arterial pulse waveform. The waveform is drawn from the voltage change as the turbulence occurs. After a few cycles, the maximum change in voltage after amplification can be easily determined. The amplified voltage is in milli-volts (mV). A trigger gate can then be programmed to open at the mid-level of the systolic upstroke, which corresponds to say, 200 mV. The waveform allows the device to approximate when the systolic/diastolic cycle is at its highest and lowest respectively, and therefore the points at which the light beams should be emitted and measured.

Selected wavelengths for glucose absorption will be triggered. The emission of the wavelengths of light is triggered by the peak in the waveform. At the systolic stage of the pulsation, when the capillaries receive blood upon pulsation, the logic gate would open (eg. when the waveform signal is at 200 mV as explained above) and the light source 40 would send beams of absorbable light 44, 46 which will be absorbed or received by both the blood and tissue. The gate will continue to remain open until the waveform takes a dive at the end of systole and will close at the same trigger level of 200 mV during the downstroke. The usual duration is about 100-200 milliseconds.

When the trigger gate opens, it sends a signal for the diode light source to fire the light beams onto the fingernail. Both the oximeter and the light source and receptor share the same microprocessor. It also activates the sensor for the detection of absorbance of the light. This is done for say, five cycles and the readings are averaged. After measuring the absorption during the peaks, light sources 44, 46 are again triggered to obtain a baseline reading in between peaks. This represents the reading of blood sugar at the tissue, skin and all other structures, but not including the arterial blood sugar level. These readings may also be obtained for say, five cycles, and the readings are averaged. Thereafter, the unabsorbable control light source 42 is activated to obtain readings for say, another five cycles and the readings are averaged. The design of the digital gate can also be in the hardware circuitry, with the microprocessor giving the cue after the maximum and range of readings of the arterial waveform is calculated.

The computation of the blood sugar level may be as follows:

(a) The amount of glucose in the systolic cycle is directly proportional to the amount of absorbable light 44, 46 absorbed as against the unabsorbable control light source 42.

(b) The amount of glucose that is consumed by tissue would be the difference between the peak value and the trough value of absorbable light 44 & 46. This represents the effectiveness of the tissue in extracting sugar from the capillary pass. It will also represent to some degree the peripheral resistance to insulin (Type II DM). If the Index of Absorption drops despite the same blood level of glucose, it may represent a tissue resistance or insulin resistance problem.

The blood sugar levels are obtained for one to two minutes and the system is switched to idle mode. The time-interval for activation can be set in terms of minutes. The default could be set to once every five minutes.

For analysis of data, it would be prudent to capture the twenty-four hour profile of the blood sugar level. All the variations in the meals and activities of a user could then be recorded. A resulting chart may show:

(a) 24 hours of blood sugar levels in the capillaries;

(b) The amount of tissue consumption (difference between peak and trough values of the absorbable light);

(c) The average day/night readings;

(d) The 2 hour past-prandial reading;

(e) Meal times, which may be button-activated by the user.

The integration of data is achieved at the display device 30. The data received are logged and time-stamped. The alarm can be individually set for both hyperglycemia and hypoglycemia. The reader/adapter provided can then download the data and plot the data into a graph. The analysis chart can be generated either via a printer, Internet or lap-top computer.

FIG. 5 is a conceptual illustration of a second detailed embodiment of the invention wherein a light source and a receiver are on the same side of a finger.

The barrel of the light source 40 has its receptor arm perpendicular to the receptor 48. It is held firmly in position by the finger-glove (or clip) including the oximeter 20. This effectively positions the light beam (B₁) at 45° to the fingernail surface. The light beam (B₁) may comprise two wavelengths of light as discussed in the previous embodiment to increase accuracy. The optimal range of the angle of contact (α₁) is between 10° to 60°. In the preferred embodiment, the receptor arm is also angled at 45° to the surface.

The light source 40 passes from A through a first lens 52 to produce a focused beam of pin-point coherent light. The intensity of the beam has been pre-set. When the beam (B₁) strikes the nail surface 24, an initial reflection will occur at B₂, while some of the beam continues to strike the nail bed where the capillaries lie. At this juncture, when the effective systolic cycle is at its peak (triggering the gate), the capillaries are filled. B₁ striking the blood column at this time will result in some of the beam being absorbed by glucose. The rest will be reflected as B₃.

As B₂ and B₃ travel up the receptor arm 48, they will pass through a second convex lens 54 which will focus and re-unite the 2 beams before they reach the sensor of the receptor 48. The change in intensity of light after reflection/absorption is registered for comparison to the source at A. A second source of light will act as control in that it will be fired from A with the same intensity as the assigned one. However, the wavelength of the control light beam approximates to 9,000 nm. At this wavelength, the absorption by glucose is very insignificant, and relatively more of the control light will be reflected.

Therefore, whatever distortion or loss in intensity of the control beam will be due to the inherent tissue properties. By comparing the differences in the intensities of the absorbable light and non-absorbable light, it is possible to calculate the amount of absorbance of light due to the presence of glucose at the pre-capillary end of the blood vessels as previously described.

Wavelength of Light Used

The invention can be performed using any wavelengths that will penetrate the skin or reflected by a fingernail, as appropriate. Preferably, the wavelength used is between 1,500 nm and 2,400 nm, as it has been found to be fairly effective in penetrating the fingernail bed to the capillary bed, and to be absorbed by the tissue and blood glucose.

In a particularly preferred embodiment, two wavelengths are used, one at 1,500 nm and the other at 2,400 nm. This is to find the maximum absorption of the combination of wavelengths in the capillary blood. This combination will enhance the signal, giving a more faithful amplification and conversion.

The optimal wavelength of the light source may be produced by using a pure single wavelength laser beam generated by a diode. A gate shutter is used to control the pulses of light emitting from the source to the nail bed. This is controlled digitally by the “gate” mechanism and timed according to the arterial waveform signal generated by the pulse oximeter. FIG. 7 is an example of such an arterial waveform.

Control of Gate Mechanism

The waveform signal corresponding to the systolic wave is determined by the oximeter. After stabilization for some time (about one minute), the logic gate is established to open when the peak value is reached. The logic gate is opened for the light source 40 to send the light beams 42, 44, 46 for measurement at a pre-determined point (say, 200 mV) at the upstroke of a systolic cycle. The logic gate will close at the end of each systolic cycle at a pre-determined point (say, 200 mV again) when the waveform dips.

The wavelengths of absorbable light 44, 46 are fixed and similar predetermined intensities of both will be generated when the logic gate opens. The value is sent back to the display device 30 (setting the peak value). Reliance is placed on the ventral/pulp side of the finger to register the signal. The signal is transmitted to the display device 30.

The light source 40 fires an impulse at the trough period and again, the value of the absorbable light 44,46 received is captured. The control light 42 (eg. having a wavelength that is more than 9,000 nm) is fired subsequent to the firing of both the absorbable light beams 44,46. The control light 42 has a wavelength not easily absorbed by glucose. The signals from the different light sources are captured for the calculation of the amount of absorbable light absorbed by glucose.

The analogue values will be converted to blood sugar levels using the formulation in the software. Values are time-stamped and stored after they undergo software filtering. Furthermore, alarm levels can be individually set if this option is included.

The Calibrator

FIG. 8 is a sample calibrator usable with the invention.

The calibrator is preferably made of a resin with a specified and predetermined absorption of wavelength of a specified absorbance value. This will correspond generally to a certain composition of glucose (95-115 mg %). The surface of the calibrator has the same consistency as the fingernail, with its overall shape preferably similar to that of a stump of the finger.

The calibrator is useful in checking the operational range of the system and acts as a counter-check when values obtained are grossly out of range.

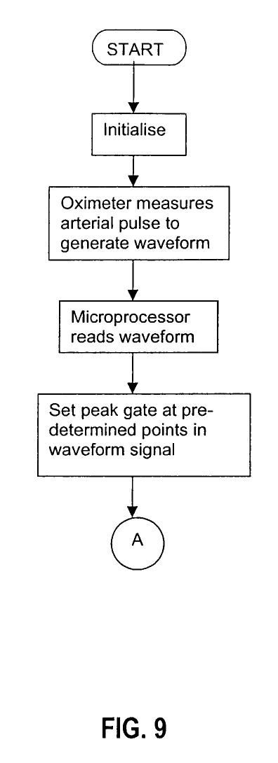

FIG. 9 is a flowchart showing the procedure of using an oximeter to determine the peak logic gate when the blood-sugar levels in the artery or capillary are at their highest.

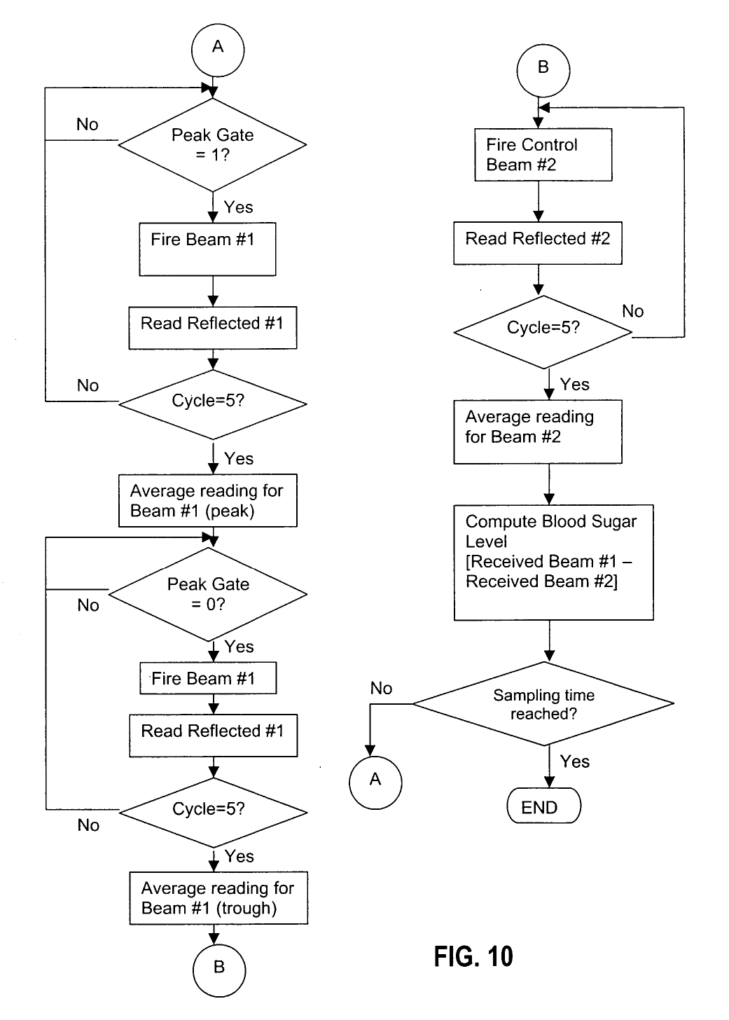

FIG. 10 is a flowchart showing the procedure for obtaining readings from the absorption of light beams.

Data Analysis of the Unabsorbed Light Beams

The light absorption data is initially passed through an amplifier for the electrical signals to be amplified. This is then passed through an analogue-to-digital converter for the readings to be converted into digital form. Following this, a low-frequency filter at the hardware circuitry level enables the interference due to noise level of, say below 8 Hz, to be filtered. Data is time-stamped and stored in the EPROM located in the display device after being processed by a microprocessor.

While a particular embodiment of the invention has been shown and described, it will be obvious to those skilled in the art that changes and modifications of the present invention may be made without departing from the invention in its broader aspects. As such, the scope of the invention should not be limited by the particular embodiment and specific construction described herein but should be defined by the appended claims and equivalents thereof. Accordingly, the aim in the appended claims is to cover all such changes and modifications as fall within the spirit and scope of the invention.

1. A device for measuring blood sugar level in vivo, comprising means to generate a waveform signal derived from the systolic and diastolic cycle in an artery or capillary, and means to trigger a measurement of blood sugar level in the artery or capillary by non-invasive means in accordance with the waveform signal.

2. A device according to claim 1, wherein the means to generate a waveform signal corresponding to the systolic and diastolic cycle comprises an oximeter.

3. A device according to claim 1 or claim 2, wherein the trigger means is set to trigger a measurement of blood sugar level when the waveform signal is at its highest and lowest as determined by the oximeter.

4. A device according to any preceding claim, wherein the non-invasive measurement of blood sugar level is performed by measuring the absorption of selected wavelengths of light transmitted by a light source.

5. A device according to claim 4, wherein the light source is adapted to transmit light at two wavelengths capable of being absorbed by blood sugar.

6. A device according to claim 5, wherein the light source is adapted to transmit light at two wavelengths at or between 1500 nm and 2400 nm.

7. A device according to any of claims 2 to 6, wherein the or each light source comprises a diode.

8. A device according to any of claims 2 to 7, including a light source adapted to transmit light at a control wavelength.

9. A device according to any preceding claim, including a display device to display the blood sugar level.

10. A device according to claim 9, wherein the display device comprises a watch.

11. A device according to any preceding claim, adapted for use on a finger or toe of a user.

12. A device according to any of claims 2 to 11, wherein the oximeter is a transmissive oximeter.

13. A device according to any of claims 2 to 11, wherein the oximeter is a reflective oximeter.

14. A device according to any one of the preceding claims, wherein the device measures arterial blood.

15. A method of measuring blood sugar level in vivo, comprising generating a waveform signal derived from the systolic and diastolic cycle in an artery or capillary of a subject and triggering measurement of blood sugar level in the artery or capillary in accordance with the waveform signal by non-invasive means.

16. A method according to claim 15, wherein the step of generating a waveform signal is performed with an oximeter.

17. A method according to claim 15 or 16, wherein the non-invasive means comprises measuring the absorption of selected wavelengths of light.

18. A method according to any of claims 15 to 17, including the steps of triggering measurement of blood sugar level against a control when the waveform signal is at its highest, then triggering measurement of blood sugar level against a control when the waveform signal is at its lowest and calculating the difference between the values obtained.

19. A method of measuring blood sugar level in vivo, comprising the use of a device as claimed in any of claims 1 to 13.
